(19) United States
(12) Patent Application Publication
(10) Pub. No.: US 2025/0099361 A1
(43) Pub. Date: Mar. 27, 2025

(54) OIL-IN-WATER EMULSION COSMETIC

(71) Applicant: SHISEIDO COMPANY, LTD., Chuo-ku, Tokyo (JP)

(72) Inventor: Kazuki MASUDA, Tokyo (JP)

(73) Assignee: SHISEIDO COMPANY, LTD., Chuo-ku, Tokyo (JP)

(21) Appl. No.: 18/833,682

(22) PCT Filed: Feb. 14, 2023
(86) PCT No.: PCT/JP2023/005051

§ 371 Date: Jul. 26, 2024

(30) Foreign Application Priority Data
Feb. 28, 2022 (JP) 2022-029037

(51) Int. Cl. A61K 8/81; A61K 8/06; A61K 8/25; A61K 8/37; A61K 8/73; A61K 8/92; A61Q 17/04

(52) U.S. Cl. CPC A61K 8/8111; A61K 8/062; A61K 8/25; A61K 8/375; A61K 8/73; A61K 8/922; A61Q 17/04; A61K 2800/262; A61K 2800/48

(57) ABSTRACT

An objective of the invention is to provide an oil-in-water emulsion cosmetic with a transparent to translucent appearance, that can stably and finely emulsify even a relatively large amount of a polar oil with a small amount of a surfactant. The oil-in-water emulsion cosmetic according to the present invention is characterized by containing (A) a non-ionic surfactant that has an HLB of 8 to 12; (B) a polar oil that is liquid at 25° C. and that has an IOB of 0.1 or higher; and (C) a water-soluble solvent; wherein the (A)/(B) ratio is 0.4 to 3.

### TECHNICAL FIELD

The present invention relates to an oil-in-water emulsion cosmetic. More specifically, the present invention relates to an oil-in-water emulsion cosmetic with a transparent to translucent appearance, that can stably and finely emulsify a polar oil with a small amount of a surfactant.

### BACKGROUND ART

Oil-in-water emulsion bases are preferred as makeup bases that can obtain a watery feeling in use because the external phase (continuous phase), in other words, the phase that first touches the skin, is the water phase. There are cases in which an oil component must be stably blended in order to provide various functions/effects and to dissolve poorly soluble components while also making use of the properties of such oil-in-water emulsion bases. However, in the case in which the oil component contains a polar oil, there is a problem in that stickiness tends to occur and the emulsion stability tends to become lower.

As a general method for stably blending large amounts of oils, increasing the amount of surfactants in accordance with the amount of oils can be contemplated. However, when the amount of surfactants is increased, there are cases in which the burden on the skin increases and safety is reduced, and in which stickiness occurs after application.

Therefore, as a method for stably blending oils, particularly polar oils, a means of improving the stability of an oil-in-water emulsion base by refining the emulsified particle size has been proposed.

For example, Patent Document 1 describes that a fine emulsion of an ester oil or a vegetable oil can be stably produced by blending, at a prescribed mass ratio, polyoxyethylene hydrogenated castor oil, to which an average of 20 to 30 moles of ethylene oxide has been added, with at least one of sorbitan monoisostearate and sorbitan diisostearate. Additionally, the oil-in-water emulsion composition obtained as a result thereof is described as being transparent to translucent, having high emulsion stability, with the average particle size of the emulsified particles being 300 nm or less.

However, with this method, it is necessary to combine and use two specific types of surfactants, i.e., polyoxyethylene hydrogenated castor oil and sorbitan mono- or diisostearate, at a specific mass ratio, thereby largely restricting the range of formulations. Additionally, there are cases in which a thickener is blended in order to provide the cosmetic with a suitable viscosity from the viewpoint of improving the texture and the ease of application, preventing dripping, etc. However, if a thickener such as carbomer is blended into a fine emulsion in which the average particle size of the emulsified particles is small, there are problems such as not being able to achieve sufficient thickening, reducing the stability and causing the appearance of the cosmetic to become milky.

### RELATED ART

### Patent Documents

Patent Document 1: JP 2021-155371 A

### SUMMARY OF THE INVENTION

### Problem to be Solved by the Invention

The present invention was made in consideration of the above-mentioned circumstances, and an objective thereof is to provide an oil-in-water emulsion cosmetic with a transparent to translucent appearance, that can stably and finely emulsify even a relatively large amount of a polar oil with a small amount of a surfactant.

### Means for Solving the Problem

The inventors carried out diligent investigations towards solving the above-mentioned problem, as a result of which they discovered that the above-mentioned problem can be solved by blending a specific non-ionic surfactant, a polar oil and a water-soluble solvent, and by setting the ratio between the non-ionic surfactant and the polar oil to be a prescribed ratio, thereby completing the present invention.

That is, the gist of the present invention is an oil-in-water emulsion cosmetic containing:
(A) a non-ionic surfactant that has an HLB of 8 to 12;
(B) a polar oil that is liquid at 25° C. and that has an IOB of 0.1 or higher; and
(C) a water-soluble solvent;
wherein the (A)/(B) ratio is 0.4 to 3.

### Effects of the Invention

In the oil-in-water emulsion cosmetic of the present invention, by having the above-mentioned features, a relatively large amount of the (B) polar oil can be stably blended with a small amount of the (A) non-ionic surfactant. Since the blended amount of the (A) non-ionic surfactant is small, the safety to the skin is high and stickiness does not tend to occur after application.

Furthermore, the oil-in-water emulsion cosmetic is provided with a fine emulsified particle size and can maintain transparency and translucency. Therefore, it can be applied to a wide range of products.

Additionally, in the case in which it is to be used as a sunscreen cosmetic in particular, a large amount of an ultraviolet absorbing agent that corresponds to the (B) polar oil or an ultraviolet absorbing agent that can be dissolved in the (B) polar oil can be blended, and therefore, a cosmetic having both a watery texture in use and high ultraviolet protection effects can be realized. Furthermore, sufficiently high ultraviolet protection effects can be realized even without blending an ultraviolet scattering agent. Therefore, the problem of unnatural whiteness caused by ultraviolet scattering agents can also be avoided.

### Modes for Carrying Out the Invention

The oil-in-water emulsion cosmetic of the present invention will be explained in detail below.

Hereinafter, there are cases in which polyoxyethylene is abbreviated to “POE”, polyoxypropylene is abbreviated to “POP” and polyethylene glycol is abbreviated to “PEG”.

### <(A) Non-Ionic Surfactant>

The (A) non-ionic surfactant (hereinafter sometimes referred to simply as “component (A)”) used in the oil-in-water emulsion cosmetic of the present invention has an HLB of 8 or higher, preferably of 9 or higher. Additionally, the upper limit of the HLB is 12 or lower, and is preferably 11 or lower. The HLB in the present invention is the weighted average for all of component (A).

Component (A) is preferably a polyoxyalkylene-based surfactant, specific examples of which include polyoxyalkylene hydrogenated castor oils (polyoxyethylene hydrogenated castor oil, etc.), polyoxyalkylene glyceryl fatty acid esters (PEG-20 glyceryl isostearate, etc.), polyoxyalkylene alkyl ethers (PEG-20 oleyl ether, PEG-10 behenyl ether, PEG-20 behenyl ether, PEG-30 behenyl ether, etc.), polyoxyalkylene sorbitan fatty acid esters (PEG-20 sorbitan monolaurate, PEG-20 sorbitan monostearate, PEG-20 sorbitan mono-oleate, etc.), polyoxyalkylene phytosterols (PEG-10 phytosterol, PEG-30 phytosterol, etc.), polyoxyalkylene-modified silicones (PEG-12 dimethicone, etc.), PPG-20 decyltetradeceth-10, PPG-13 decyltetradeceth-24, sucrose fatty acid esters (sucrose stearate), etc.

Among the above, component (A) is preferably composed of only polyoxyethylene hydrogenated castor oil (hereinafter referred to as “POE hydrogenated castor oil”), particularly POE hydrogenated castor oil in which the average number of moles of POE added is 20 to 30, since it is highly safe to the skin and exhibits particularly excellent emulsion stability with respect to the (B) polar oil. In this case, the “average number of moles of POE added” refers not only to the number of moles of POE added to one type of POE hydrogenated castor oil, but to the average number of moles of POE added to the respective types of POE hydrogenated castor oil among two or more types of POE hydrogenated castor oil that are mixed. For example, the average number of moles of POE added to a POE hydrogenated castor oil that is a 2:3 (mass ratio) mixture of a POE hydrogenated castor oil to which 20 moles of POE are added and a POE hydrogenated castor oil to which 30 moles of POE are added is (20×2+30×3)/(2+3)=26.

Commercially available products that are POE hydrogenated castor oils include POE (20) hydrogenated castor oil (HLB 10, NIKKOL HCO-20), POE (30) hydrogenated castor oil (HLB 11, NIKKOL HCO-30), etc. (both manufactured by Nikko Chemicals Co., Ltd.), and EMALEX HC-30 (manufactured by Nihon Emulsion Co., Ltd.).

The blended amount of the (A) non-ionic surfactant is preferably 1% by mass or more, more preferably 3% by mass or more, even more preferably 5% by mass or more, and preferably 30% by mass or less, more preferably 20% by mass or less, and even more preferably 10% by mass or less relative to the overall amount of the oil-in-water emulsion cosmetic. Thus, the blended amount range of the (A) non-ionic surfactant may be, for example, 1% to 30% by mass, 3% to 20% by mass, 5% to 10% by mass, etc.

### <(B) Polar Oil>

The (B) polar oil (hereinafter sometimes referred to simply as “component (B)”) used in the oil-in-water emulsion cosmetic of the present invention is an oil that is liquid at 25° C. and that preferably has an IOB value of 0.1 or higher, more preferably an IOB value higher than 0.1 and equal to or lower than 0.8.

The IOB value is an abbreviation for Inorganic/Organic Balance (inorganic/organic ratio), and is a value expressing the ratio of an inorganic value to an organic value, which indicates the degree of polarity of an organic compound. The IOB value is specifically expressed by IOB value=inorganic value/organic value. Regarding the “inorganic value” and the “organic value” respectively, an “inorganic value” and an “organic value” are set for various types of atoms or functional groups. For example, the “organic value” of a single carbon atom in a molecule is 20 and the “inorganic value” of a single hydroxyl group is 100. By summing the “inorganic values” and the “organic values” of all atoms and functional groups in an organic compound, the JOB value of that organic compound can be calculated (see, for example, Yoshio Koda, Yuki Gainenzu—Kiso to Oyo—[Organic Conceptual Diagrams-Fundamentals and Applications], pp. 11-17, Sankyo Shuppan, 1984).

Examples of component (B) satisfying these conditions include:
fatty acids such as oleic acid (IOB value=0.42) and isostearic acid (IOB value=0.43);
ester oils such as isopropyl myristate (IOB value=0.18), octyl palmitate (IOB value=0.13), isopropyl palmitate (JOB value=0.16), butyl stearate (IOB value=0.14), hexyl laurate (IOB value=0.17), myristyl myristate (IOB value=0.11), decyl oleate (IOB value=0.11), isononyl isononanoate (IOB value=0.20), isotridecyl isononanoate (IOB value=0.15), cetyl ethylhexanoate (IOB value=0.13), glycol distearate (IOB value=0.16), glyceryl diisostearate (IOB value=0.29), neopentyl glycol dicaprate (IOB value=0.25), diisostearyl malate (IOB value=0.28), trimethylolpropane triisostearate (IOB value=0.16), glyceryl tri (2-ethylhexanoate) (triethylhexanoin) (IOB value=0.35), trimethylolpropane trioctanoate (IOB value=0.33), diisobutyl adipate (IOB value=0.46), N-lauroyl-L-glutamic acid 2-octyldodecyl ester (IOB value=0.29), 2-hexyldecyl adipate (IOB value=0.16), diisopropyl sebacate (IOB value=0.40), ethylhexyl methoxycinnamate (IOB value=0.28) and ethylhexyl salicylate (IOB value=0.60);
vegetable oils such as olive oil (IOB=0.16), macadamia nut oil (IOB=0.17) and castor oil (IOB=0.42);
higher alcohols such as decyl tetradecanol (IOB=0.21), octyl dodecanol (IOB=0.26) and oleyl alcohol (IOB=0.28); and
silicone oils such as diphenylsiloxy phenyl trimethicone (IOB=0.16); etc.

In particular, when forming a sunscreen cosmetic, some or all of component (B) may be an ultraviolet absorbing agent in order to realize high ultraviolet protection effects. The ultraviolet absorbing agents satisfying the conditions for component (B) can be selected from those that are conventionally used in cosmetics, specific examples of which include octyl methoxycinnamate (2-ethylhexyl para-methoxycinnamate), ethylhexyl salicylate, homosalate, octocrylene, etc.

The blended amount of the (B) polar oil is preferably 0.1% by mass or more, more preferably 1% by mass or more, even more preferably 5% by mass or more, and preferably 40% by mass or less, more preferably 35% by mass or less, and even more preferably 30% by mass or less relative to the overall amount of the oil-in-water emulsion cosmetic. Thus, the blended amount range of the (B) polar oil may be, for example, 0.1% to 40% by mass, 1% to 35% by mass, 5% to 30% by mass, etc.

### <(C) Water-Soluble Solvent>

The (C) water-soluble solvent (hereinafter sometimes referred to simply as “component (C)”) blended into the oil-in-water emulsion cosmetic of the present invention is preferably an alcohol typically having 1 to 3 hydroxyl groups, particularly having a solubility of 10 g or more in 100 g of water at 20° C. Specifically, a lower alcohol, a polyhydric alcohol or a block-type alkylene oxide derivative is preferably used. The (C) water-soluble solvent does not contain water.

Lower alcohols include ethanol, propanol, isopropyl alcohol, butanol, etc.

Polyhydric alcohols include ethylene glycol, propylene glycol, 1,3-butylene glycol, 1,4-butylene glycol. 1,2-pentanediol (pentylene glycol), 1,5-pentanediol, neopentyl glycol, 1,2-hexanediol, 1,6-hexanediol, 1,2-cyclohexanediol, heptanediol, 1,8-octanediol, 1,9-nonanediol, 1,10-decanediol, glycerin, pentaethryritol, diethylene glycol, dipropylene glycol, triethylene glycol, tetraethylene glycol, polyethylene glycol, polypropylene glycol, thiodiglycol, etc.

Additionally, block-type alkylene oxide derivatives include dimethyl ethers of random or block copolymers of polyoxyethylene and polypropylene, such as POE (9) POP (2) dimethyl ether, POE (14) POP (7) dimethyl ether, POE (10) POP (10) dimethyl ether, POE (6) POP (14) dimethyl ether, POE (7) POP (14) dimethyl ether and POE (17) POP (4) dimethyl ether (the numbers in parentheses after POE and POP represent the respective numbers of moles added).

In particular, component (C) is preferably of one or more types selected from among lower alcohols.

The blended amount of the (C) water-soluble solvent is preferably 1% by mass or more, more preferably 3% by mass or more, even more preferably 5% by mass or more, and preferably 40% by mass or less, more preferably 30% by mass or less, and even more preferably 20% by mass or less relative to the overall amount of the oil-in-water emulsion cosmetic. Thus, the blended amount range of the (C) water-soluble solvent may be, for example, 1% to 40% by mass, 3% to 30% by mass, 5% to 20% by mass, etc.

### <(A)/(B) Ratio>

In the oil-in-water emulsion cosmetic of the present invention, the ratio of the blended amount (percent by mass) of the (A) non-ionic surfactant with respect to the blended amount (percent by mass) of the (B) polar oil is within the range 0.4 to 3, more preferably within the range 0.4 to 2.5, and even more preferably within the range 0.5 to 2. If the (A)/(B) ratio is lower than 0.4. fine emulsification becomes difficult and the transparency, the stability and the feel in use tend to be inadequate. If the ratio is higher than 3, the amount of the surfactant becomes relatively large, and there is thus a tendency for the feel in use, such as stickiness, to become poor and for the ultraviolet protection effects to become weak.

### <Optional Components>

In the oil-in-water emulsion cosmetic of the present invention, other components that are normally blended into cosmetics may be optionally blended, within a range not reducing the effects of the present invention, in addition to the aforementioned essential components (A), (B) and (C). Examples include water, oils (other than component (B)), thickeners having hydrophobic groups, ionic surfactants, ultraviolet protecting agents, powder components, humectants, various types of medicinal components, preservatives, antioxidants, etc.

### <(D) Thickener Having Hydrophobic Groups>

The thickener that can be blended into the oil-in-water emulsion cosmetic of the present invention should preferably be, in particular, (D) a thickener having hydrophobic groups (hereinafter sometimes referred to simply as “component (D)”). In the present invention, a “thickener having hydrophobic groups” means a thickener having a structure in which hydrophobic groups have been introduced to the chains of a water-soluble polymer, including copolymers, cross-polymers, etc. including at least one type of monomer having hydrophobic groups. Additionally, “having hydrophobic groups” means having long-chain alkyl groups, the long-chain alkyl groups preferably having 8 or more, more preferably 12 or more carbon atoms, and preferably 36 or fewer, more preferably 24 or fewer carbon atoms.

By blending the (D) thickener having hydrophobic groups, the oil-in-water emulsion cosmetic of the present invention can be sufficiently thickened without reducing the transparency or stability, and at the same time, a unique jiggling texture and a sense of collapsing at the time of application can be provided. The expressions “jiggling texture” and “jiggly texture” refer to having an appropriate level of resistance against a load when the cosmetic is pressed with a finger to apply a load, and after the finger has been removed, becoming still after undergoing attenuating vibrations. Meanwhile, a “sense of collapsing” at the time of application means that, when the magnitude of a load on the cosmetic when applied to the skin exceeds a limit, the shape largely changes and the cosmetic has a texture as if collapsing all at once.

Component (D) includes, for example:
acrylic polymers such as (acrylates/alkyl (C10-30) acrylate) cross-polymer, (acrylates/steareth-20 methacrylate) copolymer, (ammonium acryloyl dimethyl taurate/beheneth-25 methacrylate) cross-polymer and polyacrylate-1 (vinyl pyrrolidone/N,N-dimethylaminoethyl methacrylate/stearyl acrylate/tripropylene glycol diacrylate copolymer);
hydrophobically modified alkyl celluloses such as stearoxy hydroxypropyl methylcellulose; and
hydrophobically modified polyether urethanes such as (PEG-240/decyl tetradeceth-20/HDI) copolymer; etc.

Among the above, in particular, the hydrophobically modified polyether urethane represented by the following formula (I) is preferred:

R¹—{(O—R²)ₖ—OCONH—R³[—NHCOO—(R⁴—O)ₙ—R⁵]ₕ}ₘ (I)

In formula (I) above, R¹, R² and R⁴ each independently represent hydrocarbon groups with 2 to 4 carbon atoms. They are preferably alkyl groups or alkylene groups having 2 to 4 carbon atoms.

R³ represents a hydrocarbon group having 1 to 10 carbon atoms, possibly having a urethane bond.

R⁴ represents a hydrocarbon group having 8 to 36, preferably 12 to 24 carbon atoms.

The symbol m represents a number equal to or greater than 2, and is preferably 2. The symbol h represents a number equal to or greater than 1, and is preferably 1. The symbol k represents a number from 1 to 500, and is preferably a number from 100 to 300. The symbol n represents a number from 1 to 200, and is preferably a number from 10 to 100.

A specific example of a hydrophobically modified polyether urethane is (PEG-240/decyl tetradeceth-20/HDI) copolymer (a copolymer represented by formula (I) above, where R′ is an ethyl group, R² and R⁴ are each ethylene groups, R³ is a hexamethylene group, R$ is a 2-decyltetradecyl group, h=1, m=2, k=120 and n=20).

The blended amount of the (D) thickener having hydrophobic groups is preferably 0.05% by mass or more, more preferably 0.1% by mass or more, even more preferably 0.2% by mass or more, and preferably 5% by mass or less, more preferably 4% by mass or less, and even more preferably 3% by mass or less relative to the overall amount of the oil-in-water emulsion cosmetic. Thus, the blended amount range of the (D) thickener having hydrophobic groups may be, for example, 0.05% to 5% by mass, 0.1% to 4% by mass, 0.2% to 3% by mass, etc.

Thickeners not having hydrophobic groups should preferably not be blended because there are cases in which the stability and transparency of the oil-in-water emulsion cosmetic of the present invention can be reduced, or a sufficient viscosity or feel in use cannot be obtained. Thickeners not having hydrophobic groups include, for example, carbomer, xanthan gum, (dimethylacrylamide/sodium acryloyldimethyl taurate) cross-polymer, (ammonium acryloyldimethyl taurate/VP) copolymer, etc.

### <(E) Amino Acid-Based Ionic Surfactant>

The ionic surfactant that may be blended into the oil-in-water emulsion cosmetic of the present invention is preferably, in particular, an (E) amino acid-based ionic surfactant (hereinafter sometimes referred to simply as “component (E)”). In the present invention, an “amino acid-based ionic surfactant” refers to an anionic surfactant comprising a fatty acid (such as lauric acid and stearic acid), an amino acid (such as glutamic acid, aspartic acid, glycine and alanine, and also including free amino acid-like substances such as taurine) and an alkaline agent (such as potassium, sodium and triethanolamine).

In the oil-in-water emulsion cosmetic of the present invention, the stability and the transparency of the appearance are further improved by blending the (E) amino acid-based ionic surfactant.

Particularly favorable specific examples of component (E) include:
N-acyl methyl taurine salts such as sodium methyl myristoyl taurate, sodium methyl palmitoyl taurate, sodium methyl stearoyl taurate, sodium methyl oleoyl taurate and sodium methyl cocoyl taurate;
N-acyl glutamic acid salts such as sodium lauroyl glutamate, triethanolamine lauroyl glutamate, sodium myristoyl glutamate, sodium stearoyl glutamate, disodium stearoyl glutamate, potassium cocoyl glutamate and triethanolamine cocoyl glutamate; and
N-acyl glycine salts such as potassium cocoyl glycine, sodium cocoyl glycine, sodium lauroyl glycine and sodium lauroyl methyl glycine. Of the above, N-acyl methyl taurine salts and N-acyl glutamic acid salts are particularly preferred.

The blended amount of the (E) amino acid-based ionic surfactant is preferably 0.005% by mass or more, more preferably 0.01% by mass or more, even more preferably 0.05% by mass or more, and preferably 2% by mass or less, more preferably 1.5% by mass or less, and even more preferably 1% by mass or less relative to the overall amount of the oil-in-water emulsion cosmetic. Thus, the blended amount range of the (E) amino acid-based ionic surfactant may be, for example, 0.005% to 2% by mass, 0.01% to 1.5% by mass, 0.05% to 1% by mass, etc.

Surfactants other than amino acid-based ionic surfactants should preferably not be blended because there are cases in which the stability and transparency of the oil-in-water emulsion cosmetic of the present invention can be reduced. Such surfactants include, for example, sorbitan isostearate, etc.

### <(F) Ultraviolet Protecting Agents>

In the case in which the oil-in-water emulsion cosmetic of the present invention is intended to be a sunscreen cosmetic, aside from ultraviolet absorbing agents that correspond to the above-mentioned (B) polar off, ultraviolet absorbing agents that dissolve in the (B) polar oil and that are solid at 25° C.′, or components that enhance the effects of the ultraviolet absorbing agent (hereinafter referred to as “ultraviolet protection power enhancement components”) should preferably be blended in order to improve the ultraviolet protection power.

Oil-soluble ultraviolet absorbing agents include, for example, t-butyl methoxydibenzyolmethane, ethylhexyl triazone, diethylamino hydroxybenzoyl hexyl benzoate, bis-ethylhexyloxyphenol methoxyphenyl triazine, oxybenzone-3, methylene bis-benzotriazolyl tetramethylbutylphenol, etc.

Ultraviolet protection power enhancement components include, for example, silica powders, oil-phase thickeners such as dextrin palmitate and glyceryl (behenate/eicosanedioate), and waxes such as carnauba wax and candelilla wax. Among the above, a silica powder is most preferable from the aspect of having high ultraviolet protection power enhancement effects and also having excellent transparency of appearance.

By blending these ultraviolet protection power enhancement components, sufficient ultraviolet protection effects can be achieved with a small amount of ultraviolet absorbing agents. Thus, further suppression of stickiness can be expected.

As the silica powder, one having a particle size of 0.3 to 30 μm, preferably 0.4 to 20 μm, and more preferably 0.5 to 10 μm can be used. In this case, “particle size” is a value obtained by measurements using a particle size distribution analyzer based on laser diffraction/scattering. As the silica powder, one having surfaces that have been subjected to a hydrophobization treatment can be used. Commercially available products that are such silica powders include, for example, Godd Ball E-6C, Godd Ball B-6C (the above from Suzuki Yushi Industrial Corp.), Sunsphere L-51 (AGC Si-Tech Co., Ltd.), etc.

Additionally, commercially available products that are oil-phase thickeners that can enhance the ultraviolet protection power include Rheopearl TT (Chiba Flour Milling Co., Ltd.) and Nomcort HK-G (The Nisshin Oillio Group), etc.

The oil-in-water emulsion cosmetic of the present invention can be finely emulsified at ambient temperature and ambient pressure due to having the above-mentioned features. For this reason, compared to conventional fine emulsions requiring treatments at high temperature and high pressure, as described, for example, in JP 2016-88868 A, the amount of energy consumption necessary for production can be suppressed, and the burden on the environment can be lowered.

### <Emulsified Particle Size>

The emulsified particle size of the oil-in-water emulsion cosmetic of the present invention should preferably be smaller than 200 nm, and more preferably smaller than 100 mmm. If the emulsified particle size is smaller than 200 nm, excellent stability and a transparent to translucent appearance can be realized. The “emulsified particle size” in the present invention is a value measured by dynamic light scattering, and more specifically is a value measured by using a Zetasizer Nano (manufactured by Malvem Panalytical Ltd.) at 25° C.

### <Transparency>

In the present invention, “transparent to translucent” means a state in which, when a tubular transparent container is filled with a sample and visually observed, the sample is transparent to slightly cloudy, without being milky.

### <Stability>

The oil-in-water emulsion cosmetic of the present invention does not lose the transparent appearance even when stored for 1 month at 25° C. or for 1 week at 60° C., and thus has excellent stability with respect to temperature and over time. Furthermore, even when shaken for 1 hour at 25° C., no changes in the appearance, such as clouding or phase separation, occur. Thus, it also has excellent stability with respect to shaking.

### <Utility>

The oil-in-water emulsion cosmetic of the present invention can stably and finely emulsify a polar oil with a small amount of a surfactant and has a transparent to translucent appearance. Thus, it can be widely applied to skin cosmetics, for example, skin-care cosmetics, sunscreen cosmetics, etc. taking advantage of these characteristics.

### EXAMPLES

Though the present invention will be explained in further detail by providing examples below, the present invention is not limited in any way by these examples. Where not specially noted, the blended amounts are indicated in percentage by mass relative to the systems in which the relevant components are blended. Before specifically explaining the respective examples, the evaluation methods that were employed will be explained.

### <Transparency>>

The transparency of the appearance immediately after preparation of a sample was visually observed at 25° C.

### (Evaluation Criteria)

A: Transparent
B: Slight cloudiness (translucent)
C: Milky

### <Stability to Temperature and Over Time>

The transparency of a sample stored for 1 week at 60° C. was compared with a sample stored for 1 week at 25° C. to visually check how the transparency changed.

### (Evaluation Criteria)

A: No change
B: Some milkiness
C: Milky or separated

### <Stability to Shaking>

The transparency of a sample shaken for 1 hour at 25° C. was compared with a sample that was not shaken to visually check how the transparency changed.

### (Evaluation Criteria)

A: No change
B: Some milkiness
C: Milky or separated

### <Emulsified Particle Size>

The emulsified particle size of a prepared sample was measured by using a Zetasizer Nano (manufactured by Malvern Panalytical Ltd.) at 25° C.

### (Evaluation Criteria)

A: Smaller than 100 mm
B: 100 nm or larger and smaller than 200 nm
C: 200 nm or larger

### Examples 1 to 4 and Comparative Example 1

The components indicated in Table 1 below were mixed at ambient temperature and ambient pressure to respectively prepare oil-in-water emulsion cosmetics (samples). The emulsified particle size, the transparency, the stability with respect to temperature and over time, and the stability with respect to shaking of the respective obtained samples were evaluated in accordance with the evaluation methods described above.

TABLE 1

| | | | | | Comp. |
|---|---|---|---|---|---|
| | Ex. 1 | Ex. 2 | Ex. 3 | Ex. 4 | Ex. 1 |
| POE(20) hydrogenated | 3 | 2 | 1 | — | 1.3 |
| castor oil (HLB: 10) | | | | | |
| POE(30) hydrogenated | 1 | 2 | 3 | 4 | — |
| castor oil (HLB: 11) | | | | | |
| POB(50) hydrogenated | — | — | — | — | 2.7 |
| castor oil (HLB: 13.5) | | | | | |
| Octyl methoxycinnamate | 4 | 4 | 4 | 4 | 4 |
| (IOB: 0.27) | | | | | |
| Dipropylene glycol | 5 | 5 | 5 | 5 | 4 |
| Purified water | bal | bal | bal | bal | bal |
| Total | 100 | 100 | 100 | 100 | 100 |
| Weighted average HLB | 10.25 | 10.5 | 10.75 | 11 | 12.3 |
| (A)/(B) ratio | 1 | 1 | 1 | 1 | 1 |
| Emulsified particle size | A | A | A | A | A |
| Transparency | B | B | B | B | B |
| Stability to temperature/ | B | A | A | B | C |
| over time (60° C., 1 week) | | | | | |
| Stability to shaking | A | A | A | B | C |

As indicated in Table 1 above, the samples containing (A) a non-ionic surfactant having an HLB of 8 to 12. (B) a polar oil and (C) a water-soluble solvent, wherein the (A)/(B) ratio was within the range from 0.4 to 3, exhibited excellent results in all evaluation categories (Examples 1 to 4).

In contrast therewith, in the case in which a non-ionic surfactant having an HLB of 12.3 was used instead of the (A) non-ionic surfactant, the stability was poor (Comparative Example 1).

### Examples 5 and 6, and Comparative Examples 2 and 3

Oil-in-water cosmetics (samples) were prepared by mixing, by a conventional method, the components indicated in Table 2 below, aside from the (PEG-240/decyl tetradeceth-20/HDI) copolymer, which is the (D) thickener having hydrophobic groups, then further adding the (D) (PEG-240/decyl tetradeceth-20/HDD) copolymer, while stirring, at ambient pressure. The transparency and emulsified particle size of the respective obtained samples were evaluated by the evaluation method described above.

Additionally, the stability with respect to temperature and over time, the viscosity and the feel in use were evaluated in accordance with the evaluation methods below.

### <Stability to Temperature and Over Time>

The transparency of a sample stored for 1 month at 25° C. was compared with a sample immediately after preparation to visually check how the transparency changed.

### (Evaluation Criteria)

A: No change
B: Some milkiness
C: Milky or separated

### <Viscosity>

After storing a prepared sample for 6 hours at 30° C., a high-viscosity B-type viscometer (Rotor No. 3, rotation speed 10 rpm) was used to measure the viscosity at 30° C.

### (Evaluation Criteria)

A: 10 Pa's or higher
B: 5 Pa's or higher and lower than 10 Pas
C: Lower than 5 Pa's

### <Texture>

Expert panelists applied the prepared samples to the skin and evaluated the texture thereof.

### (Evaluation Criteria)

A: Jiggly texture and sense of collapsing when applied were clearly felt
B: Jiggly texture and sense of collapsing when applied were felt
C: Jiggly texture and sense of collapsing when applied were not felt

TABLE 2

| | Comp. | | | Comp. |
|---|---|---|---|---|
| | Ex. 2 | Ex. 5 | Ex. 6 | Ex. 3 |
| POE(10) hydrogenated castor oil | 8 | — | — | — |
| (HLB: 6.5) | | | | |
| POE(20) hydrogenated castor oil | — | 8 | — | — |
| (HLB: 10) | | | | |
| POE(30) hydrogenated castor oil | — | — | 8 | — |
| (HLB: 11) | | | | |
| POE(60) hydrogenated castor oil | — | — | — | 8 |
| (HLB: 14) | | | | |
| Octyl methoxycinnamate (IOB: 0.27) | 8 | 8 | 8 | 8 |
| Dipropylene glycol | 4 | 4 | 4 | 4 |
| (PEG-240/decyl tetradeceth-20/HDI) | 0.6 | 0.6 | 0.6 | 0.6 |
| copolymer | | | | |
| Purified water | bal | bal | bal | bal |
| Total | 100 | 100 | 100 | 100 |
| Weighted average HLB | 6.5 | 10 | 11 | 14 |
| (A)/(B) ratio | 1 | 1 | 1 | 1 |
| Transparency | B | A | A | C |
| Stability to temperature/over time | C | A | A | C |
| (25° C., 1 month) | | | | |
| Viscosity | B | A | A | C |
| Texture | C | A | A | C |
| Emulsified particle size | B | A | A | C |

As indicated in Table 2 above, the samples containing (A) a non-ionic surfactant having an HLB of 8 to 12, (B) a polar oil, (C) a water-soluble solvent and (D) a thickener having hydrophobic groups, wherein the (A)/(B) ratio was within the range from 0.4 to 3, exhibited excellent results in all evaluation categories (Examples 5 and 6). In particular, by blending the (D) thickener having hydrophobic groups, an excellent feel in use having a unique, jiggly texture and having a sense of collapsing when applied were exhibited.

In contrast therewith, in the case in which a non-ionic surfactant having an HLB of 6.5 was used instead of the (A) non-ionic surfactant, the stability with respect to temperature and over time, and the feel in use were poor (Comparative Example 2). Additionally, in the case in which a non-ionic surfactant having an HLB of 14 was used, inadequate results were exhibited in all evaluation categories (Comparative Example 3).

### Examples 7 to 12 and Comparative Example 4

The oil-in-water emulsion cosmetics having the compositions indicated in Table 3 below were prepared by a method similar to that in Example 5 above, and the transparency, the stability with respect to temperature and over time, the viscosity, the feel in use and the emulsified particle size of the respective samples were evaluated in accordance with the evaluation methods described above.

TABLE 3

| | | | | | | | Comp. |
|---|---|---|---|---|---|---|---|
| | Ex. 7 | Ex. 8 | Ex. 9 | Ex. 10 | Ex. 11 | Ex. 12 | Ex. 4 |
| POE(20) hydrogenated castor oil (HLB: 10) | 4 | 4 | 4 | 4 | 4 | 4 | 4 |
| POE(30) hydrogenated castor oil (HLB: 11) | 4 | 4 | 4 | 4 | 4 | 4 | 4 |
| Diphenylsiloxy phenyl trimethicone (IOB: | 4 | — | — | — | — | — | — |
| 0.16) | | | | | | | |
| Diisopropyl sebacate (IOB: 0.4) | — | 4 | — | — | — | — | — |
| Cetyl ethylhexanoate (IOB: 0.13) | — | — | 4 | — | — | — | — |
| Octocrylene (IOB: 0.32) | — | — | — | 4 | — | — | — |
| Ethylhexyl salicylate (IOB: 0.67) | — | — | — | — | 4 | — | — |
| Homosalate (IOB: 0.63) | — | — | — | — | — | 4 | — |
| Liquid paraffin (IOB: 0) | — | — | — | — | — | — | 4 |
| Dipropylene glycol | 4 | 4 | 4 | 4 | 4 | 4 | 4 |
| (PEG-240/decyl tetradeceth-20/HDI) | 0.6 | 0.6 | 0.6 | 0.6 | 0.6 | 0.6 | 0.6 |
| copolymer | | | | | | | |
| Purified water | bal | bal | bal | bal | bal | bal | bal |
| Total | 100 | 100 | 100 | 100 | 100 | 100 | 100 |
| Weighted average HLB | 10.5 | 10.5 | 10.5 | 10.5 | 10.5 | 10.5 | 10.5 |
| (A)/(B) ratio | 2 | 2 | 2 | 2 | 2 | 2 | 2 |
| Transparency | A | A | A | A | A | A | C |
| Stability to temperature/over time | B | A | A | A | A | A | C |
| (25° C., 1 month) | | | | | | | |
| Viscosity | A | A | A | A | A | A | C |
| Texture | A | A | A | A | A | A | C |
| Emulsified particle size | A | A | A | A | A | A | C |

As indicated in Table 3 above, in the case in which the (B) polar oil was used, adequately excellent results were exhibited in all evaluation categories (Examples 7 to 12). In contrast therewith, in the case in which liquid paraffin, which is a non-polar oil, was used instead of the (B) polar oil, inadequate results were exhibited in all evaluation categories (Comparative Example 4).

### Examples 13 to 16

The oil-in-water emulsion cosmetics having the compositions indicated in Table 4 below were prepared by a method similar to that in Example 5 above, and the transparency, the stability with respect to temperature and over time, the viscosity, the feel in use and the emulsified particle size of the respective samples were evaluated in accordance with the evaluation methods described above. The solid ultraviolet absorbing agent was pre-mixed with octyl methoxycinnamate, which is a (B) polar oil, and dissolved by heating at 90° C.

TABLE 4

| | Ex. 13 | Ex. 14 | Ex. 15 | Ex. 16 |
|---|---|---|---|---|
| POE(20) hydrogenated castor oil | 4 | 4 | 4 | 4 |
| (HLB: 10) | | | | |
| POE(30) hydrogenated castor oil | 4 | 4 | 4 | 4 |
| (HLB: 11) | | | | |
| Octyl methoxycinnamate (IOB: 0.27) | 7 | 7 | 7 | 7 |
| bis-Ethylhexyloxyphenol | 1 | — | — | — |
| methoxyphenyl triazine | | | | |
| Diethylamino hydroxybenzoyl hexyl | — | 1 | — | — |
| benzoate | | | | |
| t-Butyl methoxydibenzyolmethane | — | — | 1 | — |
| Ethylhexyl triazine | — | — | — | 1 |
| Dipropylene glycol | 4 | 4 | 4 | 4 |
| (PEG-240/decyl tetradeceth-20/HDI) | 0.6 | 0.6 | 0.6 | 0.6 |
| copolymer | | | | |
| Purified water | bal | bal | bal | bal |
| Total | 100 | 100 | 100 | 100 |
| Weighted average HLB | 10.5 | 10.5 | 10.5 | 10.5 |
| (A)/(B) ratio | 1 | 1 | 1 | 1 |
| Transparency | A | A | A | A |
| Stability to temperature/over time | A | A | A | A |
| (25° C., 1 month) | | | | |
| Viscosity | A | A | A | A |
| Texture | A | A | A | A |
| Emulsified particle size | A | A | A | A |

Even when a solid ultraviolet absorbing agent was dissolved in the (B) polar oil, the viscosity and feel in use were not worsened, and adequately excellent results were exhibited in all evaluation categories (Examples 13 to 16). By mixing a solid ultraviolet absorbing agent into the (B) polar oil, both a watery feel in use and high ultraviolet protection effects were able to be realized.

### Examples 17 to 26 and Comparative Examples 5 and 6

The oil-in-water emulsion cosmetics having the compositions indicated in Table 5 below were prepared by a method similar to that in Example 5 above, and the transparency, the stability with respect to temperature and over time, the viscosity, the feel in use and the emulsified particle size of the respective samples were evaluated in accordance with the evaluation methods described above.

TABLE 5

| | Ex. | Ex. | Ex. | Ex. | Ex. | Ex. | Ex. | Ex. | Ex. | Ex. | Comp. | Comp. |
|---|---|---|---|---|---|---|---|---|---|---|---|---|
| | 17 | 18 | 19 | 20 | 21 | 22 | 23 | 24 | 25 | 26 | Ex. 5 | Ex. 6 |
| POE(20) hydrogenated | 4 | 4 | 4 | 4 | 4 | 4 | 2 | 2 | 4 | 4 | 4 | 4 |
| castor oil (HLB: 10) | | | | | | | | | | | | |
| POE(30) hydrogenated | 4 | 4 | 4 | 4 | 4 | 4 | 2 | 2 | 4 | 4 | 4 | 4 |
| castor oil (HLB: 11) | | | | | | | | | | | | |
| PEG-10 phytosterol (HLB: | | | | | | | 2 | | | | | |
| 12.5) | | | | | | | | | | | | |
| PPG-20 decyl tetradeceth- | — | — | — | — | — | — | — | 2 | — | — | — | — |
| 10 (HLB: 9) | | | | | | | | | | | | |
| Behenyl alcohol | — | — | — | — | — | — | — | — | — | — | — | 1 |
| Stearyl alcohol | — | — | — | — | — | — | — | — | — | — | — | 1 |
| Octyl methoxycinnamate | 4 | 4 | 4 | 4 | 4 | 4 | 4 | 4 | 4 | 4 | 4 | 8 |
| (IOB: 0.27) | | | | | | | | | | | | |
| Dipropylene glycol | 4 | 4 | 4 | 4 | 4 | 4 | 4 | 4 | 4 | 4 | 4 | 5 |
| (PEG-240/decyl | 0.4 | 1 | — | — | — | — | 0.4 | 0.4 | — | 0.4 | — | 0.6 |
| tetradeceth-20/HDI) | | | | | | | | | | | | |
| copolymer | | | | | | | | | | | | |
| (Acrylates/steareth-20 | — | — | 1 | — | — | — | — | — | — | — | — | — |
| methacrylate) copolymer | | | | | | | | | | | | |
| (Ammonium acryloyl | — | — | — | 1 | — | — | — | — | — | — | — | — |
| dimethyl taurate/beheneth- | | | | | | | | | | | | |
| 25 methacrylate) cross- | | | | | | | | | | | | |
| polymer | | | | | | | | | | | | |
| (Acrylates/alkyl(C10-30) | — | — | — | — | 0.2 | — | — | — | — | — | — | — |
| acrylate) cross-polymer | | | | | | | | | | | | |
| Stearoxy hydroxypropyl | — | — | — | — | — | 2 | — | — | — | — | — | — |
| methylcellulose | | | | | | | | | | | | |
| Xanthan gum | — | — | — | — | — | — | — | — | — | — | 0.1 | — |
| Polyacrylate-1 | — | — | — | — | — | — | — | — | 1 | 0.4 | — | — |
| Triethanolamine | — | — | s.a. | — | s.a. | — | — | — | — | — | — | — |
| Lactic acid | — | — | — | — | — | — | — | — | s.a. | s.a. | — | — |
| Purified water | bal | bal | bal | bal | bal | bal | bal | bal | bal | bal | bal | bal |
| Total | 100 | 100 | 100 | 100 | 100 | 100 | 100 | 100 | 100 | 100 | 100 | 100 |
| Weighted average HLB | 10.5 | 10.5 | 10.5 | 10.5 | 10.5 | 10.5 | 11.2 | 10 | 10.5 | 10.5 | 10.5 | 10.5 |
| (A)/(B) ratio | 2 | 2 | 2 | 2 | 2 | 2 | 1.5 | 1.5 | 1 | 1 | 2 | 1 |
| Transparency | A | A | A | B | B | B | B | B | A | A | B | B |
| Stability to temperature/ | A | A | A | A | A | A | B | B | A | A | A | C |
| over time (25° C., 1 month) | | | | | | | | | | | | |
| Viscosity | A | A | A | A | A | B | A | A | A | A | C | B |
| Texture | A | A | B | A | A | B | A | A | A | A | C | C |
| Emulsified particle size | A | A | A | A | A | A | B | B | A | A | A | C |

As indicated in Table 5 above, in the case in which the (D) thickener having hydrophobic groups was used, adequately excellent results were exhibited in all evaluation categories (Examples 17 to 26). In contrast therewith, in the cases in which xanthan gum or a higher alcohol that is solid at 25° C., which are widely used as thickeners or as viscosity adjusters in cosmetics, were used instead of the (D) thickener having hydrophobic groups, inadequate results were exhibited in one of the evaluation categories (Comparative Examples 5 and 6).

### <Examples 27 and 28, and Comparative Examples 7 and 8>>

The oil-in-water emulsion cosmetics having the compositions indicated in Table 6 below were prepared by a method similar to that in Example 5 above, and the transparency, the stability with respect to temperature and over time, the viscosity, the feel in use and the emulsified particle size of the respective samples were evaluated in accordance with the evaluation methods described above.

TABLE 6

| | Comp. | | | Comp. |
|---|---|---|---|---|
| | Ex. 7 | Ex. 27 | Ex. 28 | Ex. 8 |
| POE(20) hydrogenated castor oil | 4 | 4 | 4 | 15 |
| (HLB: 10) | | | | |
| POE(30) hydrogenated castor oil | 4 | 4 | 4 | 15 |
| (HLB: 11) | | | | |
| Octyl methoxycinnamate | 24 | 16 | 4 | 6 |
| (IOB: 0.27) | | | | |
| Ethanol | 4 | 4 | — | 4 |
| Dipropylene glycol | — | — | 4 | — |
| (PEG-240/decyl tetradeceth- | 0.6 | 0.6 | 0.4 | 0.6 |
| 20/HDI) copolymer | | | | |
| Purified water | bal | bal | bal | bal |
| Total | 100 | 100 | 100 | 100 |
| Weighted average HLB | 10.5 | 10.5 | 10.5 | 10.5 |
| (A)/(B) ratio | 0.25 | 0.50 | 2.00 | 5.00 |
| Transparency | C | B | A | A |
| Stability to temperature/over time | C | B | A | A |
| (25° C., 1 month) | | | | |
| Viscosity | A | A | A | C |
| Texture | C | A | A | C |
| Emulsified particle size | C | A | A | A |

As indicated in Table 6 above, in the case in which the (A)/(B) ratio was within the range from 0.4 to 3, adequately excellent results were exhibited in all evaluation categories (Examples 27 and 28).

In contrast therewith, in cases in which the (A)/(B) ratio was outside the above range, inadequate results were exhibited in one of the evaluation categories (Comparative Examples 7 and 8).

### Examples 29 to 33

The oil-in-water emulsion cosmetics having the compositions indicated in Table 7 below were prepared by a method similar to that in Example 5 above, and the transparency, the stability with respect to temperature and over time, the viscosity, the feel in use and the emulsified particle size of the respective samples were evaluated in accordance with the evaluation methods described above. Furthermore, the ultraviolet protection power increase rate (UV protection effect boost rate) was evaluated by the method below.

### <UV Protection Effect Boost Rate>

The respective samples were dripped, at a rate of 2 mg/cm², onto measurement plates (S plates) (5×5 cm V-groove PMMA (polymethyl methacrylate) plates, SPFMASTER-PA01), spread with a finger and dried for 15 minutes, then the absorbances of the formed coating films were measured using a Hitachi U-3500 self-recording spectrophotometer. The absorbances (Abs) were computed, with an uncoated plate as the control, by using the following equation, and the measurement values were integrated from 280 nm to 400 nm to determine the absorbance integral values.

$$Abs = - log ( T / To )$$

T: transmittance of sample, To: transmittance of uncoated plate

From the absorbance integral values of the samples that were determined, the UV protection effect boost rates with reference to a control in which an ultraviolet protection power enhancement component was not blended (sample of Example 20) were computed by the following equation.

$$[ UV protection effect boost rate ( \% ) ] = [ Absorbance integral value of sample ] / [ Absorbance integral value of control ] \times 100$$

The computed UV protection effect boost rates were assessed in accordance with the evaluation criteria below.

### (Evaluation Criteria)

+++: UV protection effect boost rate of 150% or more
++: UV protection effect boost rate of 130% or more and lower than 150%+
+: UV protection effect boost rate lower than 130%

TABLE 7

| | Ex. 29 | Ex. 30 | Ex. 31 | Ex. 32 | Ex. 33 |
|---|---|---|---|---|---|
| POE(20) hydrogenated | 7 | 7 | 7 | 7 | 7 |
| castor oil (HLB: 10) | | | | | |
| POE(30) hydrogenated | 7 | 7 | 7 | 7 | 7 |
| castor oil (HLB: 11) | | | | | |
| Octyl methoxycinnamate | 20 | 20 | 20 | 20 | 20 |
| (IOB: 0.27) | | | | | |
| Ethanol | 10 | 10 | 10 | 10 | 10 |
| (PEG-240/decyl tetradeceth- | 0.4 | 0.4 | 0.4 | 0.4 | 0.4 |
| 20/HDI) copolymer | | | | | |
| Silica | — | 1 | 3 | — | — |
| Dextrin palmitate | — | — | — | 0.5 | — |
| Glyceryl (behenate/ | — | — | — | — | 0.5 |
| eicosanedioate) | | | | | |
| Purified water | bal | bal | bal | bal | bal |
| Total | 100 | 100 | 100 | 100 | 100 |
| Weighted average HLB | 10.5 | 10.5 | 10.5 | 10.5 | 10.5 |
| (A)/(B) ratio | 0.7 | 0.7 | 0.7 | 0.7 | 0.7 |
| Transparency | A | B | B | B | B |
| Stability to temperature/over | A | A | A | A | A |
| time (25° C., 1 month) | | | | | |
| Viscosity | A | A | A | A | A |
| Texture | A | A | A | A | A |
| Emulsified particle size | A | A | A | A | A |
| UV protection effect boost | Ctrl. | ++ | +++ | + | + |
| rate | | | | | |

As indicated in Table 7 above, by further adding an ultraviolet protection power enhancement component to the oil-in-water emulsion cosmetic serving as the control (Example 29), the ultraviolet protection effects were able to be markedly increased with almost no worsening in the transparency, the stability with respect to temperature and over time, the viscosity, the feel in use and the emulsified particle size (Examples 30 to 33).

### Examples 34 to 41 and Comparative Examples 9 to 11

The components indicated in Table 8 below were mixed at ambient temperature and ambient pressure to respectively prepare oil-in-water emulsion cosmetics (samples). The emulsified particle size, the transparency and the stability of the respective obtained samples were evaluated in accordance with evaluation methods similar to those for Example 1 above.

TABLE 8

| | Comp. | Ex. | Ex. | Ex. | Ex. | Ex. | Ex. | Ex. | Ex. | Comp. | Comp. |
|---|---|---|---|---|---|---|---|---|---|---|---|
| | Ex. 9 | 34 | 35 | 36 | 37 | 38 | 39 | 40 | 41 | Ex. 10 | Ex. 11 |
| POE(10) hydrogenated | 4 | 2 | — | — | — | — | — | — | — | — | — |
| castor oil (HLB: 6.5) | | | | | | | | | | | |
| POE(20) hydrogenated | — | 2 | 4 | 2 | — | — | 2 | 2 | 2 | 1 | 1 |
| castor oil (HLB: 10) | | | | | | | | | | | |
| POE(30) hydrogenated | — | — | — | 2 | 4 | 2 | — | 2 | 2 | — | — |
| castor oil (HLB: 11) | | | | | | | | | | | |
| POE(40) hydrogenated | — | — | — | — | — | 2 | — | — | — | — | — |
| castor oil (HLB: 12.5) | | | | | | | | | | | |
| POE(50) hydrogenated | — | — | — | — | — | — | 2 | — | — | 3 | 3 |
| castor oil (HLB: 13.5) | | | | | | | | | | | |
| Octyl methoxycinnamate | 4 | 4 | 4 | 4 | 4 | 4 | 4 | 4 | 4 | 4 | 4 |
| (IOB: 0.27) | | | | | | | | | | | |
| Ethanol | — | — | — | — | — | — | — | — | — | — | 5 |
| Dipropylene glycol | 5 | 5 | 5 | 5 | 5 | 5 | 5 | 5 | 5 | 5 | — |
| Sodium cocoyl methyl | 0.1 | 0.1 | 0.1 | 0.1 | 0.1 | 0.1 | 0.1 | — | — | 0.1 | 0.1 |
| taurate | | | | | | | | | | | |
| Potassium cocoyl | — | — | — | — | — | — | — | 0.2 | — | — | — |
| glutamate | | | | | | | | | | | |
| Sodium cocoyl glycine | — | — | — | — | — | — | — | — | 0.2 | — | — |
| Purified water | bal | bal | bal | bal | bal | bal | bal | bal | bal | bal | bal |
| Total | 100 | 100 | 100 | 100 | 100 | 100 | 100 | 100 | 100 | 100 | 100 |
| Weighted average HLB | 6.5 | 8.25 | 10 | 10.5 | 11 | 11.75 | 11.75 | 10.5 | 10.5 | 12.63 | 12.63 |
| (A)/(B) ratio | 1 | 1 | 1 | 1 | 1 | 1 | 1 | 1 | 1 | 1 | 1 |
| Emulsified particle size | C | B | A | A | A | A | A | A | A | C | C |
| Transparency | C | B | A | A | A | A | B | A | A | C | C |
| Stability to temperature/ | C | B | A | A | A | B | A | A | A | C | C |
| over time (60° C., 1 week) | | | | | | | | | | | |
| Stability to shaking | C | B | A | A | A | B | A | A | A | C | C |

As indicated in Table 8 above, the samples containing (A) a non-ionic surfactant, (B) a polar oil. (C) a water-soluble solvent and (E) an amino acid-based ionic surfactant, wherein the (A)/(B) ratio was within the range from 0.4 to 3, exhibited excellent results in all evaluation categories (Examples 34 to 41). Additionally, even among those in which the transparency was ranked A, Examples 35 to 38 had particularly exceptional transparency.

In contrast therewith, in the case in which a non-ionic surfactant having an HLB of 6.5 or a non-ionic surfactant having an HLB of 12.63 was used instead of the (A) non-ionic surfactant having an HLB of 8 to 12, inadequate results were exhibited in all evaluation categories (Comparative Examples 9 to 11).

### Examples 42 to 44 and Comparative Examples 12 and 13

Oil-in-water cosmetics (samples) were prepared by mixing, by a conventional method, the components indicated in Table 9 below, aside from the (PEG-240/decyl tetradeceth-20/HDI) copolymer, which is a (D) thickener having hydrophobic groups, then further adding (PEG-240/decyl tetradeceth-20/HDI) copolymer as component (D), while stirring, at ambient pressure. The emulsified particle size, the transparency, and the stability with respect to temperature and over time of the respective obtained samples were evaluated by evaluation methods similar to those in Example 1 above, and the viscosity and the feel in use were evaluated by evaluations methods similar to those in Example 5 above.

TABLE 9

| | | | | Comp. | Comp. |
|---|---|---|---|---|---|
| | Ex. 42 | Ex. 43 | Ex. 44 | Ex. 12 | Ex. 13 |
| POE(10) hydrogenated | — | — | — | 8 | — |
| castor oil (HLB: 6.5) | | | | | |
| POE(20) hydrogenated | 4 | 4 | 1 | — | 1 |
| castor oil (HLB: 10) | | | | | |
| POE(30) hydrogenated | 4 | 4 | 8 | — | 1 |
| castor oil (HLB: 11) | | | | | |
| Octyl methoxycinnamate | 8 | 8 | 8 | 8 | 8 |
| (IOB: 0.27) | | | | | |
| Dipropylene glycol | 5 | 5 | 5 | 5 | 5 |
| (PEG-240/decyl tetradeceth- | 0.6 | 0.6 | 0.6 | 0.6 | 0.6 |
| 20/HDI) copolymer | | | | | |
| Sodium methyl stearoyl | — | — | 0.01 | 0.01 | — |
| taurate | | | | | |
| Sodium methyl cocoyl | — | 0.1 | — | — | 6 |
| taurate | | | | | |
| Phenyl benzimidazole | 1 | 1 | — | 1 | 1 |
| sulfonic acid | | | | | |
| Triethanolamine | s.a. | s.a. | s.a. | s.a. | s.a. |
| Purified water | bal | bal | bal | bal | bal |
| Total | 100 | 100 | 100 | 100 | 100 |
| Weighted average HLB | 10.5 | 10.5 | 11 | 6.5 | 10.5 |
| (A)/(B) ratio | 1 | 1 | 1 | 1 | 0.25 |
| Emulsified particle size | A | A | A | C | C |
| Transparency | B | A | A | C | C |
| Stability to temperature/ | B | A | A | C | C |
| over time (60° C., 1 week) | | | | | |
| Viscosity | B | A | A | B | B |
| Texture | A | A | A | C | C |

As indicated in Table 9 above, the sample containing (A) a non-ionic surfactant having an HLB of 8 to 12, (B) a polar oil, (C) a water-soluble solvent and (D) a thickener having hydrophobic groups, wherein the (A)/(B) ratio was within the range from 0.4 to 3, exhibited excellent results in all evaluation categories (Example 42). Additionally, it was confirmed that the transparency, the stability with respect to temperature and over time, and the viscosity improved by further blending an (E) amino acid-based surfactant (Examples 43 and 44).

In contrast therewith, in the case in which a non-ionic surfactant having an HLB of 6.5 was used instead of the (A) non-ionic surfactant and the case in which the (A)/(B) ratio was 0.25, improvements in the evaluation categories were not observed, even when an (E) amino acid-based surfactant was blended (Comparative Examples 12 and 13).

1. An oil-in-water emulsion cosmetic containing:
(A) a non-ionic surfactant that has an HLB of 8 to 12;
(B) a polar oil that is liquid at 25° C. and that has an IOB of 0.1 or higher, and
(C) a water-soluble solvent;
wherein the (A)/(B) ratio is 0.4 to 3.

2. The oil-in-water emulsion cosmetic according to claim 1, wherein the (A) non-ionic surfactant is a POE hydrogenated castor oil to which an average of 20 to 30 moles of POE has been added.

3. The oil-in-water emulsion cosmetic according to claim 1, wherein the (B) polar oil is an ultraviolet absorbing agent.

4. The oil-in-water emulsion cosmetic according to claim 1, wherein the (C) water-soluble solvent is a lower alcohol.

5. The oil-in-water emulsion cosmetic according to claim 1, wherein an emulsified particle size is smaller than 200 nm.

6. The oil-in-water emulsion cosmetic according to claim 1, which is transparent to translucent.

7. The oil-in-water emulsion cosmetic according to claim 1, further containing (D) a thickener having hydrophobic groups.

8. The oil-in-water emulsion cosmetic according to claim 1, further containing (E) an amino acid-based ionic surfactant.

9. The oil-in-water emulsion cosmetic according to claim 1, further containing an ultraviolet absorbing agent that dissolves in the (B) polar oil and that is solid at 25° C.

10. The oil-in-water emulsion cosmetic according to claim 1, further containing an ultraviolet protection power enhancement component selected from the group consisting of silica powders, dextrin palmitate, glyceryl (behenate/eicosanedioate), carnauba wax and candelilla wax.
